(19) United States
(12) Patent Application Publication
(10) Pub. No.: US 2006/0004105 A1
(43) Pub. Date: Jan. 5, 2006

(54) Pharmaceutical composition for treating stress incontinence and/or mixed incontinence

(75) Inventors: Martin Michel, Amsterdam (NL); Marion Wienrich, Weiterstadt (DE); Ursula Ebinger, Boonton Township, NJ (US)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 10/977,883

(22) Filed: Oct. 29, 2004

(30) Foreign Application Priority Data
Oct. 31, 2003 (DE) 103 51 271

(51) Int. Cl. A61K 31/195

(52) U.S. Cl. 514/567

(57) ABSTRACT

This invention describes the use of beta-3-adrenoceptor agonists for the treatment of functional bladder disorders, particularly stress incontinence and/or mixed incontinence.

This invention describes the use of beta-3-adrenoceptor agonists for the treatment of functional bladder disorders, particularly stress incontinence, and/or mixed incontinence.

### DESCRIPTION OF THE PRIOR ART

The incidence of urinary incontinence is constantly increasing as a result of changes in the ageing statistics. Nevertheless those affected are for the most part still untreated or inadequately treated. Apart from the medical consequences such as chronic infections of the urinary passages, urinary incontinence for those affected is associated with a high psychological burden of suffering. It is estimated that 100 million older people are affected by urinary incontinence.

The lower urinary tract consists of the bladder, the urethra, and the corresponding muscles and the ligaments of the suspensory apparatus. The purpose of the bladder is to store the urine and to empty it. The important factors for performing the storage function are not only the relaxation of the bladder muscle (detrusor muscle), but also the closure mechanisms provided by the neck of the bladder and the smooth muscle of the urethra and also by the cross-striated muscle of the urethra and the pelvic floor. During the emptying of the bladder (micturition), the detrusor muscle contracts while the urethra and pelvic floor relax and the sphincter muscle of the bladder opens. These processes require complex control by the parasympathetic, sympathetic, and somatic nervous system.

Functional bladder problems are a heterogeneous group of disorders which differ in their etiology, diagnosis, and therapy. In the standardizing recommendations of the International Continence Society (ICS) urinary incontinence is defined as involuntary loss of urine which is objectively detectable and constitutes a social and hygiene problem. Generally, urinary incontinence only occurs when there is an unintentional increase in the pressure in the bladder during the storage phase. This can happen as a result of unrestricted contractions of the detrusor muscle (urge incontinence) or failure of the urethral closure mechanism (stress incontinence).

According to the ICS definition, overactive bladder (OAB) is characterized by an irresistible imperative need to urinate, which may or may not be associated with urge incontinence, usually with increased frequency of micturition and nocturnal urination. Pathophysiologically, this complaint may be based on involuntary contractions during the filling phase, the cause of which may be neurogenic or non-neurogenic (idiopathic) in nature.

Urge incontinence is characterized by an irresistible urge to urinate and involuntary loss of urine.

Stress incontinence is characterized by the involuntary loss of urine which generally occurs at moments of elevated intra-abdominal pressure. This may occur for example when lifting, coughing, sneezing, running while at the same time there is no detrusor activity. Loss of urine takes place as the result of a variable combination of an insufficiency of the sphincter muscles of the bladder and the pelvic floor as well as anatomical defects in the suspensory apparatus. As a result the closure pressure of the urethra is too low and incontinence results. Pure stress incontinence often occurs in women, particularly if they have given birth. In men, this form of urinary incontinence is usually only observed after prostatectomies or other surgical interventions on the small pelvis.

In so-called mixed incontinence patients suffer from symptoms of both stress incontinence and urge incontinence. Once again, it is mainly women who are affected.

For treating the various forms of functional bladder disorders, particularly stress incontinence or mixed incontinence, various therapeutic approaches are available.

Stress incontinence is treated primarily by conservative and surgical procedures. Up till now there has been no generally suitable drug therapy available. Alpha-adrenoceptor agonists such as pseudoephedrine and phenylpropanolamine have shown some effect, albeit very modest, in the treatment of low-grade stress incontinence. A disadvantage is that they have no selectivity for the urethral muscles and have numerous side effects such as hypertension, tachycardia, arrhythmia, sleep disorders, headaches, and tremors.

The treatment of mixed incontinence is a controversial subject of discussion and comprises combinations of invasive procedures for treating the stress incontinence component and drug therapies for treating the urge incontinence component.

Since the mid-1995s, it has been reported that selective beta-3-adrenoceptor-agonists are promising in the treatment of urinary incontinence (EP 0 958 835). It has now been found that compounds of the class of the beta-3-adrenoceptor agonists promise to be highly effective while being well tolerated in the treatment of mixed incontinence. In addition, their activity should be restricted to the storage phase of the bladder and unimpeded emptying of the bladder should be guaranteed without any build-up of urine residues.

Despite the many promising approaches and progress in the treatment of stress incontinence and/or mixed incontinence, the development of efficient and well-tolerated therapies remains a challenge.

### SUMMARY OF THE INVENTION

The present invention sets out to contribute to the treatment of urinary incontinence. Preferably the invention is suitable for the treatment of stress incontinence and/or mixed incontinence.

It is proposed according to the invention to use beta-3-adrenoceptor agonists, or pharmaceutical compositions which contain compounds from this category of active substances.

### DESCRIPTION OF THE INVENTION

According to the present invention a new pharmaceutical composition is provided which contains as active ingredient at least one beta-3-adrenoceptor agonist in a pharmaceutically effective amount.

a) Active Components

In the description of the preferred embodiment certain terminology will be used hereinafter in the interests of clarity. This terminology should include the embodiment described and all technical equivalents which work in a similar manner for a similar purpose to achieve similar results. To the extent that any pharmaceutically active compound is disclosed or claimed, it is expressly intended that all active metabolites which are produced in vivo are included, and it is expressly intended that all enantiomers, diastereomers or tautomers are included, if the compound is capable of occurring in its enantiomeric, diastereomeric or tautomeric form. Obviously, the isomer which is pharmacologically most effective and most free from side effects is preferred. Also included are pharmacologically acceptable salts thereof. Examples of pharmaceutically active salts for each of the compounds which are the subject of this description include, without being restricted thereto, salts which are prepared from pharmaceutically acceptable acids or bases, including organic and inorganic acids and bases. If the preferred compound is basic, salts may be prepared from pharmaceutically acceptable acids. When selecting the most preferred salt, or to clarify whether a salt or the neutral compound is used, properties such as bioavailability, ease of manufacture, workability and shelf life are taken into consideration, inter alia. Suitable pharmaceutically acceptable acids include acetic acid, benzenesulphonic acid (besylate), benzoic acid, p-bromophenylsulphonic acid, camphorsulphonic acid, carbonic acid, citric acid, ethanesulphonic acid, fumaric acid, gluconic acid, glutamic acid, hydrobromic acid, hydrochloric acid, hydriodic acid, isethionic acid, lactic acid, maleic acid, malic acid, mandelic acid, methanesulphonic acid (mesylate), mucinic acid, nitric acid, oxalic acid, pamoic acid, pantothenic acid, phosphoric acid, succinic acid, sulphuric acid, tartaric acid, p-toluenesulphonic acid and the like. Examples of pharmaceutically acceptable salts include, without being restricted thereto, acetate, benzoate, hydroxybutyrate, bisulphate, bisulphite, bromide, butyne-1,4-dioate, caproate, chloride, chlorobenzoate, citrate, dihydrogenphosphate, dinitrobenzoate, fumarate, glycollate, heptanoate, hexyne-1,6-dioate, hydroxybenzoate, iodide, lactate, maleate, malonate, mandelate, metaphosphate, methanesulphonate, methoxybenzoate, methylbenzoate, monohydrogenphosphate, naphthalene-1-sulphonate, naphthalene-2-sulphonate, napthalene-2-sulphonate, oxalate, phenylbutyrate, phenylproprionate, phosphate, phthalate, phenylacetate, propanesulphonate, propiolate, propionate, pyrophosphate, pyrosulphate, sebacate, suberate, succinate, sulphate, sulphite, sulphonate, tartrate, xylenesulphonate, and the like.

Insofar as it is necessary for completeness, the methods of synthesis of the compounds for which the prior art is mentioned and the dosages thereof are expressly included by reference to the prior art mentioned at the corresponding point.

The beta-3-adrenoreceptor agonist used according to the invention is preferably a phenoxyacetic acid derivative. This is preferably selected from the following group: 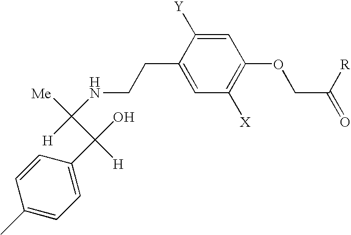
with

1) X═Br Y═H, R═OH
2-[2-bromo-4-[2-[[(1S,2R)-2-hydroxy-2-(4-hydroxyphenyl)-1-methylethyl]amino]ethyl]phenoxy]acetic acid,

2) X═Cl, Y═H, R═OH
2-[2-chloro-4-[2-[[(1S,2R)-2-hydroxy-2-(4-hydroxyphenyl)-1-methylethyl]amino]ethyl]phenoxy]acetic acid,

3) X═Y═Cl, R═OH
2-[2,5-dichloro-4-[2-[[(1S,2R)-2-hydroxy-2-(4-hydroxyphenyl)-1-methylethyl]amino]ethyl]phenoxy]acetic acid,

4) X═Y═H, R═OH
2-[4-[2-[[(1s,2R)-2-hydroxy-2-(4-hydroxyphenyl)-1-methylethyl]amino]ethyl]2,5-dimethylphenoxy]acetic acid,

5) X═OH; Y═H; R═OH
2-[2-hydroxy-4-[2-[[(1S,2R)-2-hydroxy-2-(4-hydroxyphenyl)-1-methylethyl]amino]ethyl]phenoxy]acetic acid,

6) X═Cl; Y═H, R═OEt
ethyl-2-[2-chloro-4-[2-[[(1S,2r)-2-hydroxy-2-(4-hydroxyphenyl)-1-methylethyl]amino]ethyl]phenoxy]acetate,

7) X═Cl; Y═Cl, R═OEt
ethyl-2-[2,5-dichloro-4-[2-[[(1S,2r)-2-hydroxy-2-(4-hydroxyphenyl)-1-methylethyl]amino]ethyl]phenoxy]acetate,

8) X═Me; Y═Me, R═OEt
(−)-ethyl-2-[4-(2-{[(1S,2R)-2-hydroxy-2-(4-hydroxyphenyl)-1-methylethyl]amino}ethyl)-2,5-dimethylphenyloxy]acetate,

9) X=Me; Y=Me, R=OH
(−)-2-[4-(2-{[(1S,2R)-2-hydroxy-2-(4-hydroxyphenyl)-1-methylethyl]amino}ethyl)-2,5-dimethylphenloxy]acetic acid,

Details of the above-mentioned compounds 1 to 9 can be found in WO 00/02846 and its equivalent U.S. Pat. No. 6,538,152, which are incorporated herein by reference in their entirties.

Other preferred examples of phenoxyacetic acid derivatives are:

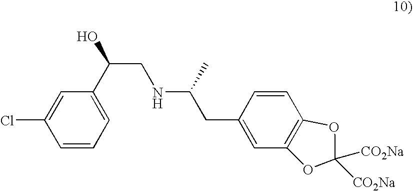

### Disodium-([R,R]-5-2-[[2-(3-chlorophenyl)-2-hydroxyethyl]-amino]propyl)-1,3-benzodioxol-2,2-dicarboxylate

More information on this substance can be found in J. Med. Chem. 2001, 44, 1456-1466, or Journal of Urology, 165(1):240-244, January 2001.

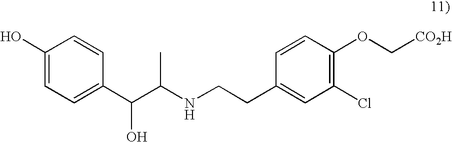

### 2-[2-chloro-4-(2-{[(1S,2R)-2-hydroxy-2-(4-hydroxyphenyl)-1-methylethyl]amino}ethyl)phenoxy]acetic acid

More information on this substance can be found in J. Med. Chem. 2003, 46, 105-112. 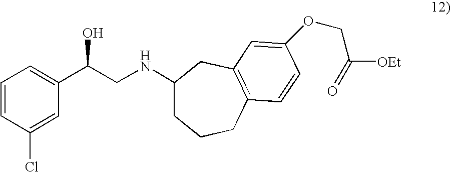
FK175

ethyl[R-(R*,S*)]-[[8-[[2-(3-chlorophenyl)-2-hydroxyethyl]amino]-6,7,8,9-tetrahydro-5H-benzocyclohepten-2-yl]oxy]-acetate, hydrochloride. 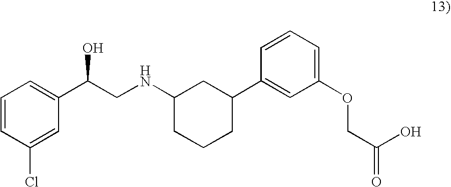
GS-332

[1S-[α,3β(S*)]]-3-[3-[[2-(3-chlorophenyl)-2-hydroxyethyl]amino]cyclohexyl]phenoxy]-acetic acid, monosodium salt,

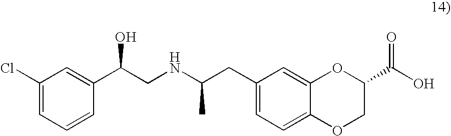

### 7-{2-[2-(3-chloro-phenyl)-2-hydroxy-ethylamino]-propyl}-2,3-dihydro-benzo[1,4]dioxine-2-carboxylic acid

More information on this compound, also know as N-5984, can be found in the literature.

As well as these compounds the following compounds selected from among the beta-3-adrenoreceptor agonists are also suitable.

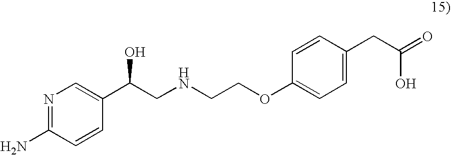

### (4-{2-[2-(6-amino-pyridin-3-yl)-2-hydroxy-ethylamino]-ethoxy}-phenyl)-acetic acid

More information on this compound, also known as CP-331684, can be found in the literature.

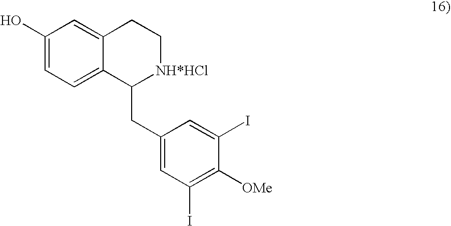

### 5-(4-methoxy-3,4-diiodobenzyl)-5,6,7,8-tetrahydro-naphthalen-2-ol

More information on this substance can be found in J. Med. Chem. 2001, 44, 1456-1466.

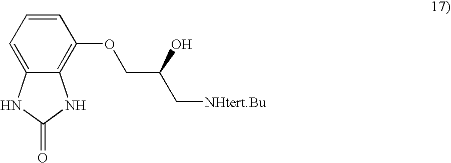

### 4-(1-N-tert.butylamino-2-hydroxy-butoxy)-1,3-dihydro-benzoimidazol-2-one

More information on this substance, which is also known as CGP 12177A, can be found in Journal of Urology, 165(1):240-244, January 2001, or in J. Med. Chem. 2001, 44, 1456-1466.

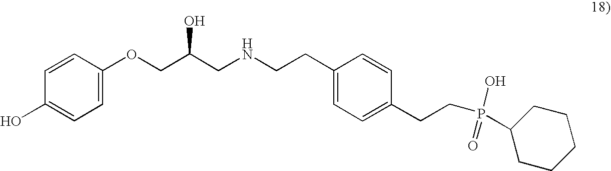

More information on this substance, which is also known as SB 226552, can be found in J. Med. Chem. 2001, 44, 1456-1466.

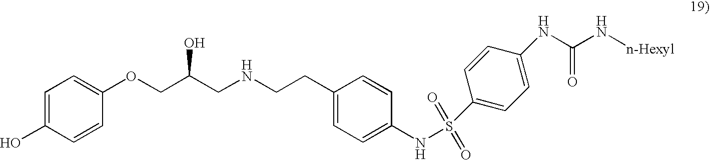

More information on this substance, which is also known as L755507, can be found in J. Med. Chem. 2001, 44, 1456-1466.

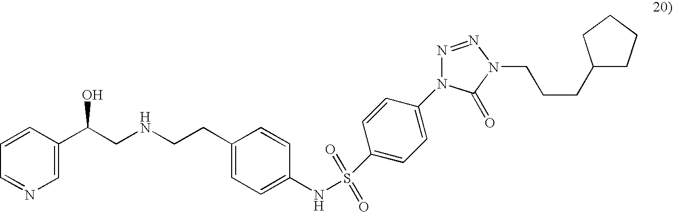

More information on this substance, which is also known as L770664, can be found in J. Med. Chem. 2001, 44, 1456-1466.

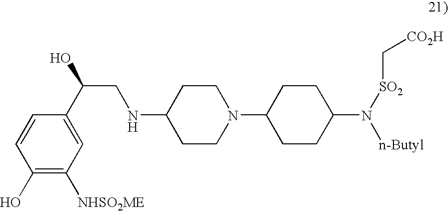

### 4-N-(4-(2-(4-hydroxy-3-methylsulphonamido-phenyl)-2-hydroxy-ethylamino)-piperidinyl)-phenyl-n-butyl-aminosulphonylacetic acid

More information on this substance can be found in J. Med. Chem. 2001, 44, 1456-1466, or in the Bioorg. Med. Chem. Lett. 9 (2001) 2045. 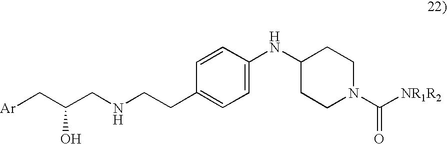
where
a) Ar=4-OHPh-O—, R1=octyl, R2=H
b) Ar=4-OH,3-methylsulphonylamidophenyl-O, R1=2,5-diFbenzyl, R2=H
c) Ar=4-OH,3-methylsulphonylamidophenyl, R1=2,5-diFbenzyl, R2=H

More information on these substances can be found in Bioorg. Med. Chem. Lett. 11 (2001) 3123-3127.

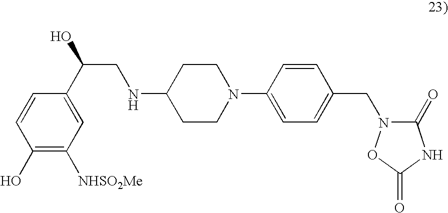

More information on this substance can be found in Bioorg. Med. Chem. Lett. 11 (2001) 981-984.

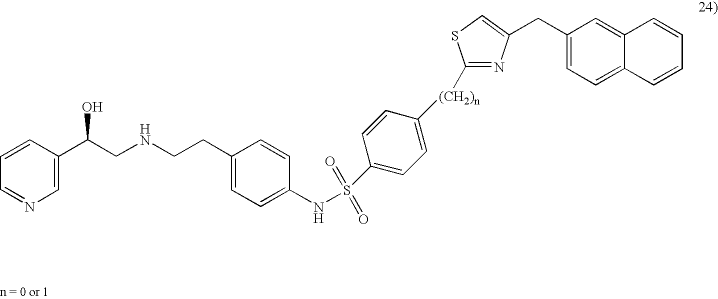

More information on this substance can be found in Bioorg. Med. Chem. Lett. 10 (2000) 1971-1973.

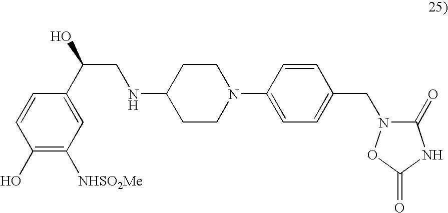

More information on this substance can be found in Bioorg. Med. Chem. Lett. 11 (2001) 757-760.

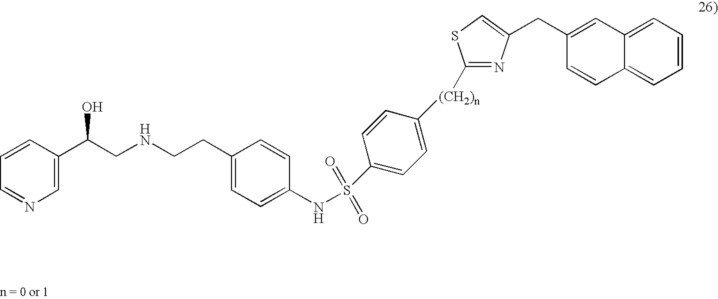

More information on this substance can be found in Bioorg. Med. Chem. Lett. 10 (2000) 1971-1973.

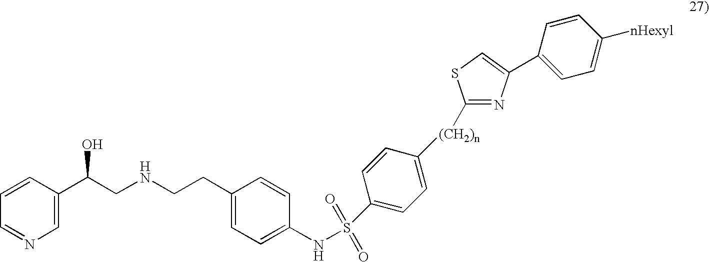

More information on this substance can be found in Bioorg. Med. Chem. Lett. 10 (2000) 1971-1973.

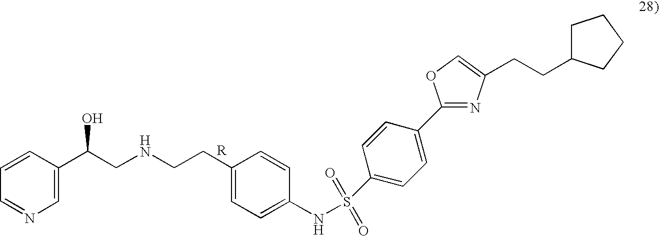

More information on this substance can be found in the Bioorg. Med. Chem. Lett. 10 (2000) 1531-1534.

29) 2-(3-{[2-(3-chlorophenyl)-2R-hydroxyl-ethylamino]ethylamino}phenyl)furan-3-carboxylic acid. More information on this compound can be found in the literature.

30) 2-(3-{[2-(3-chlorophenyl)-2R-hydroxyl-ethylamino]ethylamino}phenyl)thiophene-3-carboxylic acid. Information on this compound can be found in the literature.

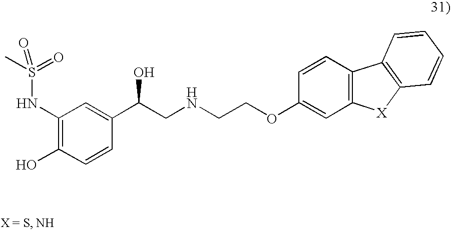

More information on this compound, also known as SB-418790, can be found in the literature.

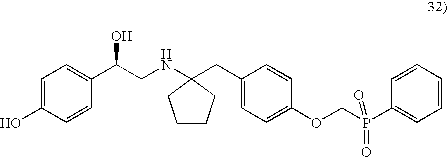

More information on this compound, also known as SB-251023, can be found in the literature.

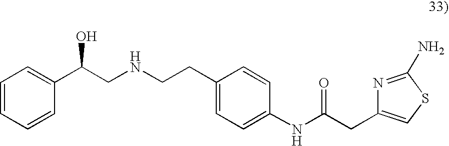

More information on this compound, (R)-2-(2-aminothiazol-4-yl)-4′-[2-[2-(hydroxy-2-phenylethyl)amino]ethyl]acetanilide, can be found in the literature WO 03/037881.

34) (S)-4-[2-hydroxy-3-[[2-[4-(5-carbamoyl-2-pyridyloxy)phenyl]-1,1-dimethyl-ethyl]amino]-propoxy]-carbazole (LY 377604).

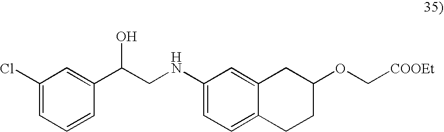

This compound is also known by the name SR 58611.

Most preferred are: (−)-ethyl-2-[4-(2-{[(1S,2R)-2-hydroxy-2-(4-hydroxyphenyl)-1-methylethyl]amino}ethyl)-2,5-dimethylphenyloxy]acetate, (−)-ethyl-2-[4-(2-{[(1S,2R)-2-hydroxy-2-(4-hydroxyphenyl)-1-methylethyl]amino}ethyl)-2,5-dimethylphenloxy]acetate-monohydrochloride, (−)-2-[4-(2-{[(1S,2R)-2-hydroxy-2-(4-hydroxyphenyl)-1-methylethyl]amino}ethyl)-2,5-dimethylphenyloxy]acetic acid, or other pharmacologically acceptable salts thereof.

Particularly interesting examples of beta-3-adrenoceptor agonists are (−)-ethyl-2-[4-(2-{[(1S,2R)-2-hydroxy-2-(4-hydroxyphenyl)-1-methylethyl]amino}ethyl)-2,5-dimethylphenyloxy]acetate or (−)-2-[4-(2-{[(1S,2R)-2-hydroxy-2-(4-hydroxyphenyl)-1-methylethyl]-amino}ethyl)-2,5-dimethylphenyloxy]acetic acid, enantiomers or other stereoisomers thereof, and pharmacologically active salts thereof.

These compounds are disclosed in WO 00/02846 or WO 2003024916.

These last two compounds are represented by the following formula II, which should take precedence over the specified name, in the event of any inconsistencies: 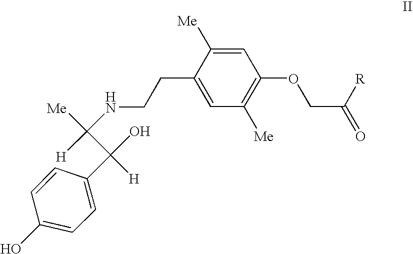
where R═O-ethyl:
(−)-ethyl-2-[4-(2-{](1S,2R)-2-hydroxy-2-(4-hydroxyphenyl)-1-methylethyl]amino}ethyl)-2,5-dimethylphenyloxy]acetate, preferably the monohydrate,
   where R═OH:
(−)-2-[4-(2-{[(1S,2R)-2-hydroxy-2-(4-hydroxyphenyl)-1-methylethyl]amino}-ethyl)-2,5-dimethylphenyloxy]acetic acid.
   b) Dosage

In order to determine the optimum dose of active substance, various basic conditions have to be taken into consideration, such as, for example, the age and body weight of the patient, the nature and stage of the disease, and the potency of the compound. This is deemed to be within the capabilities of the skilled man, and the existing literature on the components can be consulted in order to arrive at the optimum dose. The doses specified refer to the dosage after the end of the adjustment phase.

The doses given hereinafter expressly include all the numerical values, both whole numbers and fractions, within the range specified. The data relate to adults. Pediatric doses may be lower.

Doses administered more than once a day or twice a day (e.g. 3, 4, 5, or 6 times a day) are also expressly included herein.

In some cases a smaller amount than that specified may be sufficient while in other cases a larger total amount may be required.

The total daily dose may be taken in one go or in several portions depending on the treatment plan. The treatment plan may also prescribe intervals of longer than one day between the doses.

The average daily dose of the beta-3-agonist for an adult human is about 1 mg to 1000 mg, preferably 10 mg to about 750 mg per day, preferably 50 to 500 mg, more preferably 80 to 200 mg, administered in one or more doses.

c) Formulations

The compositions of the present invention may conveniently be administered in a pharmaceutical composition which contains the active components in combination with a suitable carrier. Such pharmaceutical compositions may be prepared by methods and contain carriers which are well known in the art. Generally recognized textbooks are available to the skilled man for this purpose.

The compositions of the present invention may be administered parenterally (e.g. by intravenous, intraperitoneal, subcutaneous or intramuscular injection), topically, orally, intranasally, transdermally, rectally, by pulmonary or nasal inhalation. Oral administration is particularly preferred. Of the oral formulations, those which are resistant to gastric juices may be preferred. In this case, capsules or tablets resistant to gastric juices are preferred, and in both cases this may be achieved with a coating which is resistant to gastric juices. The skilled man will find instructions for formulations resistant to gastric juices in the prior art.

Various formulating options are described below. The skilled man may choose a suitable formulation from them.

For oral therapeutic administration the composition according to the invention may be combined with one or more carriers and used in the form of tablets for swallowing, buccal tablets, sublingual tablets, sugar-coated tablets, sprays, powders, pastilles, coated tablets, granules, capsules, elixirs, suspensions, solutions, syrups, lozenges, chewing gums, foods, and the like.

A powder may be prepared for example by grinding the particles of active substance to a suitable size.

Dilute powders may be prepared by finely grinding the powdered substance with a non-toxic carrier material such as lactose and delivering it as a powder. Other suitable carrier materials for this purpose are other carbohydrates such as starch or mannitol. These powders may optionally contain flavorings, preservatives, dispersing agents, colorings, and other pharmacological adjuvants.

Capsules may be prepared from a powder of the kind described above or other powders, which are placed in a capsule, preferably a gelatin capsule, and the capsule is then sealed.

It is also possible for lubricants known from the prior art to be introduced into the capsule or used to seal the two parts of the capsule. The efficacy of a capsule when taken orally can be increased by the addition of disintegrating or solubilizing substances such as, for example, carboxymethylcelluose, carboxymethylcellulose calcium, low-substituted hydroxypropylcellulose, calcium carbonate, sodium carbonate and other substances. The active substance may be present in the capsule not only as a solid but also in suspended form, for example in vegetable oil, polyethyleneglycol, or glycerol using surface-active substances, etc.

Tablets may be prepared by compressing the powdered mixture and then processing it into granules, for example. The tablets may contain various excipients such as e.g. starches, lactose, sucrose, glucose, sodium chloride, urea for tablets for dissolving or injecting, amylose, various types of cellulose as described above and others. Glycerol or starch may be used as a moisture retaining agent.

The disintegrants used may be, for example, starch, alginic acid, calcium alginate, pectic acid, powdered agar-agar, formaldehyde gelatin, calcium carbonate, sodium bicarbonate, magnesium peroxide, and amylose.

Anti-disintegrants or solution retardants which may be used include, for example, sucrose, stearin, solid paraffin (preferably with a melting point in the range from 50-52° C.), cocoa butter, and hydrogenated fats.

Other disintegrants may be: corn starch, potato starch, alginic acid, and the like.

Suitable absorption accelerators include, inter alia, quaternary ammonium compounds, sodium lauryl sulphate, and saponins.

Ether may be used, for example, as a binder distributor and cetyl alcohol, glycerol monostearate, starch, corn starch, lactose, wetting agents (e.g., Aerosol® OT, pluronics, ands/or tweens), gum tragacanth, gum arabic, gelatin, and others may be used as hydrophilizing agents or disintegration accelerators.

Sucrose, fructose, lactose, or aspartame may be used as sweeteners while peppermint, wintergreen oil, cherry flavoring, etc. may be used as flavoring agents.

The following may also be generally used as additional excipients: fumed silica (e.g., Aerosil®), sodium dioctyl sulfosuccinate (e.g., Aerosol® OT), ethylcellulose, ion-exchange resin (e.g., Amberlite® XE-88), corn starch, Amisterol, amylose, microcrystalline-cellulose (e.g., Avicel®), bentonite, calcium sulphate, polyethylene glycol (e.g., Carbowax® 4000 and 6000), carrageen, castor wax, cellulose, microcrystalline cellulose, crospovidone, dextrane, dextrin, dicalcium phosphate, pharmaceutical tablet base, kaolin, lactose (USP), lactosil, magnesium stearate, mannitol, granular mannitol N.F. methylcellulose, fatty acid esters (e.g., Miglyol® 812 neutral oil), powdered milk, powdered sugar, nal-tab, crystalline sorbitol, povidone (e.g., Plasdone®), polyethyleneglycols, polyvinylacetate phthalate, polyvinylpyrrolidone, atomized glyceryl palmitostearate (e.g., Precirol®), neat's foot oil (hydrogenated), melting tablet base, silicone, stabiline, pregelatinized starch (e.g., Starx® 1500), silica (e.g., Syloid®), Waldhof tablet base, tablettol, talcum cetylatum and stearatum, metal soaps, fructose, and cellulose ethers (e.g., Tylose®). The tabletting excipient K (M25) is particularly suitable, and also complies with the requirements of the following pharmacopoeias: DAB, Ph. Eur., BP, and NF.

Other excipients known from the prior art may also be used.

The tablets may be produced by direct compression, for example.

It is also possible to prepare other formulations for oral administration such as solutions, syrups, elixirs, etc. If desired the compound may be micro-encapsulated.

Parenteral administration may be achieved by dissolving the compound in a liquid and injecting it by subcutaneous, intramuscular, or intravenous route. Suitable solvents include, for example, water, or oily media.

In order to prepare suppositories the compound may be formulated with low-melting and water-soluble or water-insoluble materials such as polyethylene glycol, cocoa butter, higher esters (for example, moerysthyl, palmitate), or mixtures thereof.

The above list is provided solely by way of example and a skilled man might consider other excipients.

Various other materials may be provided as coatings or for modifying the physical form of the solid dosage units in some other way. For example, tablets, pills, or capsules may be coated with gelatin, wax, shellac, or sugar and the like. As already mentioned, formulations resistant to gastric juices are preferred for the oral preparations. Therefore, gastric juice-resistant coatings are preferred for tablets or capsules. In the case of a syrup or elixir, sucrose, or fructose may be used as the sweetener, methyl- and propylparaben may be present as preservatives, and a coloring and a flavoring agent such as cherry or orange flavor may also be present.

The excipients mentioned above are not restricted to the use of the formulation in connection with which they have been mentioned but may also be applied to the other formulations.

Naturally, any material used in the preparations of any of these dosage units must be pharmaceutically acceptable and substantially non-toxic in the amounts used. In addition, the active components may be incorporated in preparations with delayed release and devices which, without being restricted thereto, include those based on osmotic pressures, in order to achieve the desired release profile. One-a-day formulations for each of the active components are particularly included.

Compositions and preparations of this kind should contain at least 0.001% of active compound. The percentage of the compositions and preparations may naturally vary and may appropriately make up between 0.1 and about 100% of the weight of a given dosage unit. The quantity of active compound in therapeutically useful compositions of this kind is such that an effective dose is present.

d) Indications

The pharmaceutical composition and each of the compounds listed as beta-3-adrenoreceptor agonists according to the invention may be used to treat or prevent, inter alia, each of the syndromes mentioned below, as an individual syndrome and in conjunction with another of the syndromes mentioned, without being restricted thereto: urinary incontinence, particularly stress incontinence, urge incontinence, mixed incontinence or overactive bladder of neurogenic or non-neurogenic origin and further sub-indications thereof.

Thus, the invention includes both those syndromes whose cause is dysfunction or disease of an organ and those which can be attributed to diseases or disorders of the central nervous system. Accordingly, every treatment of bladder function disorder, particularly urinary incontinence of all kinds, is taken into account by the present invention.

Thus, a further embodiment of the present invention comprises using the composition according to the invention to prepare a drug for treating or preventing any of the indications of bladder dysfunction mentioned in the preceding paragraph.

The above diseases or disorders are treated by administering a therapeutically effective amount of the composition according to the invention to a mammal. In most cases this is a human being but the treatment of farm animals (e.g. cattle) and domestic animals (e.g. dogs, cats and horses) is also expressly covered. For use in veterinary medicine the dosages used may be different from those specified herein.

Within the scope of the indication of overactive bladder and/or urge incontinence all the compounds are particularly preferred, apart from the compounds 1), 2), 3), 4), 5), 6), 7), 8), 9).

Within the scope of the present invention the indications of stress incontinence and/or mixed incontinence are particularly preferred, while the indication of mixed incontinence is most preferred.

It is expected that the new composition will provide rapid relief for those suffering from the above diseases and disorders with a minimum amount of harmful side effects.

What is claimed is:

1. A pharmaceutical composition comprising a beta-3-adrenoceptor agonist that treats a functional bladder disorder in a mammal, the pharmaceutical composition adapted to be administered parenterally, topically, orally, intranasally, transdermally, rectally, or by pulmonary or nasal inhalation, the beta-3-adrenoceptor being present in the pharmaceutical composition in an amount about between about 1 mg and about 1000 mg.

2. Pharmaceutical composition according to claim 1, wherein the beta-3-adrenoceptor agonist comprises a phenoxyacetic acid derivative.

3. Pharmaceutical composition according to claim 1, wherein the beta-3-adrenoceptor agonist comprises a compound selected from the group consisting of compounds 1 to 35, and metabolites, enantiomers, and a pharmacologically acceptable salts thereof.

4. Pharmaceutical composition according to claim 1, wherein the beta-3-adrenoceptor agonist comprises a compound selected from the group consisting of compounds 1 to 9, and metabolites, enantiomers, and a pharmacologically acceptable salts thereof.

5. Pharmaceutical composition according to claim 1, wherein the beta-3-adrenoceptor agonist comprises a compound selected from the group consisting of compounds 10 to 35, and metabolites, enantiomers, and a pharmacologically acceptable salts thereof.

6. Pharmaceutical composition according to claim 1, wherein the beta-3-adrenoceptor agonist comprises a compound selected from the group consisting of:
(−)-ethyl-2-[4-(2-{[(1S,2R)-2-hydroxy-2-(4-hydroxyphenyl)-1-methylethyl]amino}ethyl)-2,5-dimethylphenloxy]acetate;
(−)-2-[4-(2-{[(1S,2R)-2-hydroxy-2-(4-hydroxyphenyl)-1-methylethyl]amino}ethyl)-2,5-dimethylphenyloxy]acetic acid; and
metabolites, enentiomers, and pharmacologically acceptable salts thereof.

7. Pharmaceutical composition according to claim 1, wherein the functional bladder disorder is urinary incontinence or overactive bladder.

8. Pharmaceutical composition according to claim 1, wherein the functional bladder disorder is selected from the group consisting of: urinary stress incontinence, urinary urge incontinence, neurogenic overactive bladder, non-neurogenic overactive bladder, and combinations thereof.

9. Pharmaceutical composition according to claim 1, wherein the functional bladder disorder comprises urinary stress incontinence and urinary urge incontinence.

10. Pharmaceutical composition according to claim 1, wherein the functional bladder disorder comprises neurogenic overactive bladder or non-neurogenic overactive bladder.

11. Method of treating a functional bladder disorder in a mammal comprising: administering to a patient a pharmaceutical composition comprising beta-3-adrenoceptor agonist or a pharmaceutically effective salt thereof, the pharmaceutical composition adapted to be administered parenterally, topically, orally, intranasally, transdermally, rectally, or by pulmonary or nasal inhalation, the beta-3-adrenoceptor being present in the pharmaceutical composition in an amount about between about 1 mg and about 1000 mg.

12. Method according to claim 11, wherein the beta-3-adrenoceptor agonist comprises a phenoxyacetic acid derivative.

13. Method according to claim 11, wherein the beta-3-adrenoceptor agonist comprises a compound selected from the group consisting of compounds 1 to 35, and metabolites, enantiomers, and a pharmacologically acceptable salts thereof.

14. Method according to claim 11, wherein the beta-3-adrenoceptor agonist comprises a compound selected from the group consisting of compounds 1 to 9, and metabolites, enantiomers, and a pharmacologically acceptable salts thereof.

15. Method according to claim 11, wherein the beta-3-adrenoceptor agonist comprises a compound selected from the group consisting of compounds 10 to 35, and metabolites, enantiomers, and a pharmacologically acceptable salts thereof.

16. Method according to claim 11, wherein the beta-3-adrenoceptor agonist comprises a compound selected from the group consisting of:
(−)-ethyl-2-[4-(2-{[(1S,2R)-2-hydroxy-2-(4-hydroxyphenyl)-1-methylethyl]amino}ethyl)-2,5-dimethylphenloxy]acetate;
(−)-2-[4-(2{[(1S,2R)-2-hydroxy-2-(4-hydroxyphenyl)-1-methylethyl]amino}ethyl)-2,5-dimethylphenloxy]acetic acid; and
metabolites, enantiomers,and pharmacologically acceptable salts thereof.

17. Method according to claim 11, wherein the functional bladder disorder is urinary incontinence or overactive bladder.

18. Method according to claim 11, wherein the functional bladder disorder is selected from the group consisting of: urinary stress incontinence, urinary urge incontinence, neurogenic overactive bladder, non-neurogenic overactive bladder, and combinations thereof.

19. Method according to claim 11, wherein the functional bladder disorder comprises urinary stress incontinence and urinary urge incontinence.

20. Method according to claim 11, wherein the functional bladder disorder comprises neurogenic overactive bladder or non-neurogenic overactive bladder.
